(19) United States
(12) Patent Application Publication
(10) Pub. No.: US 2011/0054409 A1
(43) Pub. Date: Mar. 3, 2011

(54) MEDICAL LINE STRAP SECUREMENT SYSTEM

(75) Inventor: Vasu Nishtala, Snellville, GA (US)

(73) Assignee: C.R. BARD, INC., Covington, GA (US)

(21) Appl. No.: 12/548,168

(22) Filed: Aug. 26, 2009

(51) Int. Cl. A61M 25/02

(52) U.S. Cl. 604/179; 604/174

(57) ABSTRACT

A securement system is provided for use with a medical article. The securement system includes a retainer and a securement strap. The retainer comprising an elastomeric gel and a longitudinal channel configured to retain at least a portion of the medical article. In some embodiments, the securement strap may be configured to secure the retainer and retained medical article relative to a patient. A method for securing a medical article to a patient is further disclosed.

### BACKGROUND OF THE INVENTION

1. Field of the Invention

This invention relates to a retainer and a securement system employing the retainer for securing a medical article to a patient.

2. Description of the Related Art

It is common in the treatment of patients to utilize catheters to introduce fluids and medications directly into the patient or to withdraw fluids from the patient. Often, it becomes desirable to maintain such catheterization over an extended period of time during the treatment of a patient. In order to keep the catheter or other medical line property positioned for the duration of treatment, the catheter or medical line can be secured to the patient in a variety of ways. Most commonly, this involves taping the catheter or medical line to the patient.

Securing a catheter with tape upon the patient traditionally has certain drawbacks. The use of tape at the insertion site can retain dirt or other contaminant particles, potentially leading to infection of the patient. Tape also fails to limit catheter motion and, therefore, contributes to motion related complications like phlebitis, infiltration and catheter migration. Additionally, removal of taped dressings can itself cause undesired motion of the catheter upon the patient.

The use of such adhesive tape can also quite time-consuming, requiring strips of adhesive tape or similar material to be individually cut and trimmed to fasten the catheter directly to the skin of a patient. The time involved becomes a more significant factor in situations where the adhesive tape must be frequently changed. Changes in swelling conditions of the limb in which the catheter is placed exacerbate these problems as they often require re-stabilization of the inserted catheter with new adhesive. Moreover, in some circumstances, the condition of the skin may prevent proper adhesion of the adhesive strips,

As mentioned, taped dressings also require periodic changes. The frequent, often daily, removal and reapplication of adhesive tape to the skin of the patient can excoriate the skin in the area around the dressing. Such repeated applications of tape over the catheter or medical line can additionally lead to the build up of adhesive residue on the outer surface of the catheter or medical line. This residue can result in contaminants adhering to the catheter itself, increasing the likelihood of infection of the insertion site. This residue can also make the catheter or medical line stickier and more difficult to handle for healthcare providers. Further, moisture may build up where the patient's skin is covered by the tape.

A variety of securement devices for attachment of medical devices are commercially available. Such products can include means, such as hook and loop fasteners, etc., for securing a medical device, such as a catheter, etc., to a body of a patient. However, such products are still deficient with respect to complexity of manufacture and ease of use. Accordingly, there remains a need for an improved medical line securement device for use with a patient that overcomes the problems associated with current designs.

### SUMMARY OF THE INVENTION

The devices and methods disclosed herein have several features, no single one of which is solely responsible for its desirable attributes. Without limiting the scope as expressed by the claims that follow, its more prominent features will now be discussed briefly.

In one aspect, provided is a securement system for use with an elongated medical article. The securement system includes an elongated flexible securement strap having an inner surface and an outer surface, the strap being configured to wrap around a portion of a patient, at least a portion of the inner surface contacting a patient's skin. The system further includes a retainer disposed on at least a portion of the inner surface of the strap and having a channel and channel opening arranged along a longitudinal axis, at least a portion of the retainer being compressible so as to substantially match contours of the patient's skin and of the secured medical article at least when the strap is secured around the portion of the patient.

In another aspect, provided is a method of securing a medical article to a patient. The method includes providing an apparatus, the apparatus including a securement strap having an inner surface and an outer surface, and a retainer disposed on at least a portion of the inner surface of the strap and having a longitudinal channel configured to receive and retain at least a portion of the medical article. The method further includes placing at least a portion of the medical article within the longitudinal channel and securing the retainer and medical article to the patient with the securement strap so that the retainer is disposed between the patient and the securement strap and the secured portion of the medical article is inhibited from moving in the lateral, longitudinal and transverse directions relative to the patient.

In another aspect, provided is a retainer for use with an elongated medical article. The retainer includes a channel sized and shaped to receive at least a portion of the medical article and a channel opening arranged along a longitudinal axis and extending through a bottom surface of the retainer. The retainer further includes an elastomeric gel formed by curing an organopolysiloxane composition. The gel is compressible so as to substantially match contours of a patient's skin and of the medical article at least when the bottom surface is urged against the patient's skin.

In one form, the retainer comprises an elastomeric gel. In another form, the elastomeric gel is formed by curing an organopolysiloxane composition.

In still another form, the organopolysiloxane composition includes a vinyl-containing high viscosity organopolysiloxane or a blend of high viscosity vinyl-containing organopolysiloxanes, a low viscosity organopolysiloxane or a blend of low viscosity organopolysiloxanes, a reinforcing filler, a platinum catalyst and a hydrogen containing polysiloxane copolymer

In a further form, the retainer comprises memory foam. In a still further form, the memory foam comprises polyurethane.

Still other aspects, features, and advantages are readily apparent from the following detailed description, by illustrating a number of exemplary forms and implementations, including the best mode contemplated. The forms disclosed are also capable of other and different forms, and several details can be modified in various respects, all without departing from the spirit and scope of this disclosure. Accordingly, the drawings and descriptions are to be regarded as illustrative in nature, and not as restrictive.

These and other features of the present invention will become readily apparent to those skilled in the art from the following detailed description of the preferred embodiments, which refers to the attached figures. The invention is not limited, however, to the particular embodiments that are disclosed.

### BRIEF DESCRIPTION OF THE DRAWINGS

These and other features, aspects, and advantages of the invention disclosed herein are described below with reference to the drawings of preferred embodiments, which are intended to illustrate and not to limit the invention. Additionally, from figure to figure, the same reference numerals have been used to designate the same components of an illustrated embodiment. The following is a brief description of each of the drawings.

FIG. 1 is a perspective view showing a securement system according to a preferred embodiment of the present invention;

FIG. 2 is a perspective view showing the securement system from FIG. 1 in a fastened configuration;

FIG. 3 is a top view of the securement system from FIG. 1;

FIG. 4 is a side view of the securement system from FIG. 1;

FIG. 5 is a top view of the retainer shown in FIGS. 1-4;

FIG. 6 is a side view of the retainer shown in FIG. 5;

FIG. 7 is a perspective view showing a securement system according to a preferred embodiment of the present invention with a medical article affixed to a retainer;

FIG. 8 is a perspective view of the securement system from FIG. 7 in a fastened configuration;

FIG. 9 is a top view of the securement system from FIG. 7;

FIG. 10 is a side view of the securement system from FIG. 7;

FIG. 11 is a perspective view showing the securement system from FIG. 7 used to secure a medical article on a patient; and

FIG. 12 is a cross-section through the securement system, medical article, and patient taken along line 12-12 of FIG. 11.

### DETAILED DESCRIPTION OF THE PREFERRED EMBODIMENTS

The following description and examples illustrate preferred embodiments of the present securement system in detail which is disclosed in the context of use with an exemplary medical device. The principles of the present invention, however, are not limited to medical devices. It will be understood by those of skill in the art in view of the present disclosure that the securement system described can be used with medical articles, including, but not limited to, catheters, fluid delivery tubes, electrical wires, and other medical devices or their components. One skilled in the art may also find additional applications for the devices and methods disclosed herein. Thus, the illustrations and descriptions of the securement system in connection with the medical devices are merely exemplary of some possible applications of the securement system.

To assist in the description of these components of the securement system, the following coordinate terms are used. Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise. A “longitudinal axis” is generally parallel to a portion of the catheter hub, the connector fitting or other medical article retained by the securement system, as well as parallel to the axis of a channel of the retainer, through which the medical article extends. A “lateral axis” is normal to the longitudinal axis. A “transverse axis” extends normal to both the longitudinal and lateral axes. The “longitudinal direction” refers to a direction substantially parallel to the longitudinal axis; “the lateral direction” refers to a direction substantially parallel to the lateral axis; and “the transverse direction” refers to a direction substantially parallel to the transverse axis. The term “axial” as used herein refers to the axis of the channel or connector fitting, and therefore is substantially synonymous with the term “longitudinal” as used herein.

Also, the terms “proximal” and “distal”, which are used to describe the present securement system, are used consistently with the description of the exemplary applications. Thus, proximal and distal are used in reference to the insertion site into the patient's body. The terms “upper,” “lower,” “top,” “bottom,” “underside,” “upperside” and the like, which also are used to describe the present securement system, are used in reference to the illustrated orientation of the embodiment. For example, the term “upperside” is used to describe the portion of the retainer that is located above a lateral axis that passes through the axis of the channel.

The term “alkyl” refers to radicals having from 1 to 8 carbon atoms per alkyl group, such as methyl, ethyl, propyl, butyl, pentyl, hexyl, octyl and the like. The term “alkenyl” refers to radicals having from 2 to 8 carbon atoms such as, vinyl, allyl and 1-propenyl. The term “aryl” refers to mononuclear and binuclear aryl radicals such as, phenyl, tolyl, xylyl, naphthyl and the like; mononuclear aryl alkyl radicals having from zero (i.e. no alkyl group or a bond) to 8 carbon atoms per alkyl group such as benzyl, phenyl and the like. The term “monovalent hydrocarbon radicals” includes hydrocarbon radicals such as alkyl, alkenyl and aryl.

The term “tacky” means that the tacky gel pad has an adhesive property that is somewhat sticky to the touch, enabling the pad or sheet padding to be readily attached to a limb or other area of a patient's body, yet is easily removed, i.e. is releasably attached. The term “macerating” means to soften the skin over a period of time, especially as a result of the skin being wetted or occluded. The term “limb” refers to the paired appendages of the body used especially for movement or grasping, including the legs, knees, shins, ankles, feet, toes, arms, elbows, forearms, wrists, hands, fingers or any part thereof. The term “curing” refers to any process by which the raw or uncured polysiloxanes containing reinforcing agents are converted to a finished product, i.e. the soft, tacky, reinforced polysiloxane elastomer.

Various aspects will now be described with reference to specific forms or embodiments selected for purposes of illustration. It will be appreciated that the spirit and scope of the securement system disclosed herein is not limited to the selected forms. Moreover, it is to be noted that the figures provided herein are not drawn to any particular proportion or scale, and that many variations can be made to the illustrated embodiments. Brief introductions to some of the features, which are common to the described embodiments of the securement systems, are now described. Those of skill in the art will recognize that the disclosed aspects and features shown herein are not limited to any particular form of a securement system, and securement systems that include one or more of the features herein described can be designed for use with a variety of medical articles.

The forms disclosed herein provide a securement system for securing a medical article to a patient. The medical article may have an elongated body. The elongated body cooperates with a retainer to arrest movement of the medical article in longitudinal, lateral, and/or transverse directions when placed within the retainer. In each of the forms described below, the securement system has a retainer that includes a channel formed there through. The medical article is installed or removed from the channel. The retainer is disposed such that the medical article is between at least a portion of the retainer and the patient's skin. The retainer may comprise a gel pad that has little or no ability to induce maceration of the skin, due in part to its permeability for transporting water vapor from the skin through the gel pad. Such an arrangement induces little or no maceration when applied to the skin for an extended period.

The retainer may include at least one abutment that cooperates with at least one contact point or surface on the medical article. The one or more abutments of the retainer extend generally normal to the axis of the channel and can be, for example, but without limitation, a surface, a wall of a slot, a ridge, a protuberance, or like structures. The abutment cooperates with the one or more contact points or surfaces of the medical article to inhibit longitudinal movement of the medical article through the channel. For example, the abutment could be a surface on the distal end of the retainer that acts against at least a portion of an outwardly extending member of the medical article. In this way, the medical article will be limited in its proximal movement (i.e., movement toward the insertion site) once the outwardly extending member contacts or abuts against the distal end of the retainer.

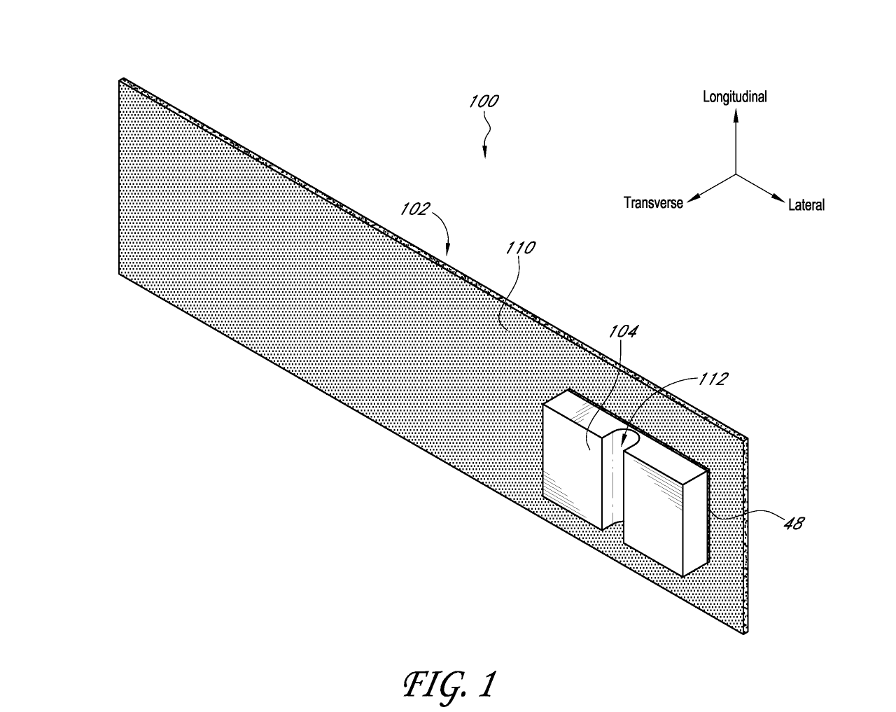

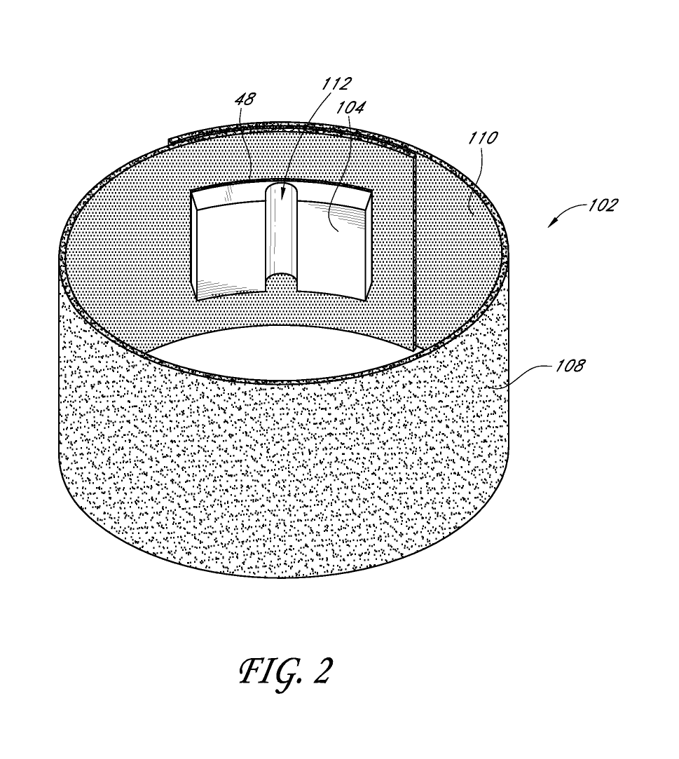

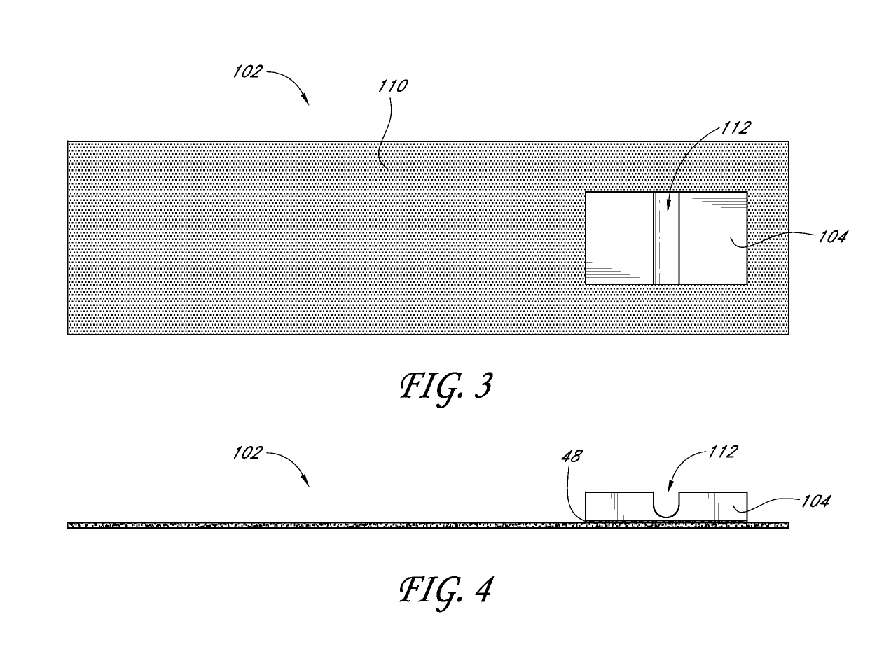

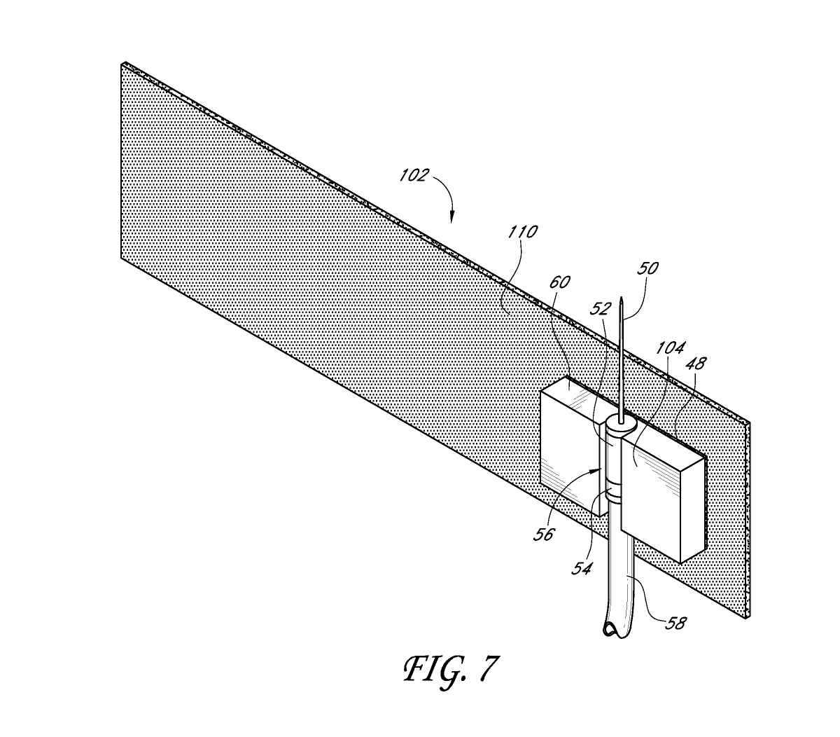

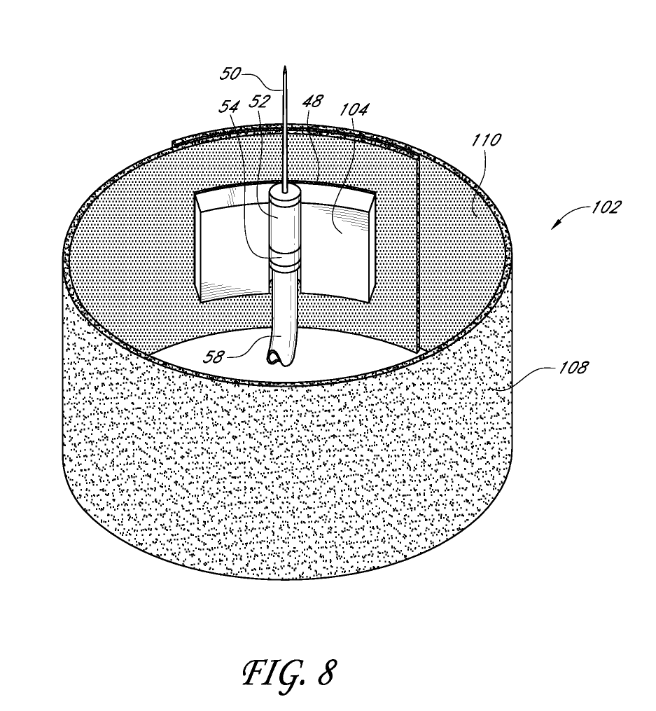

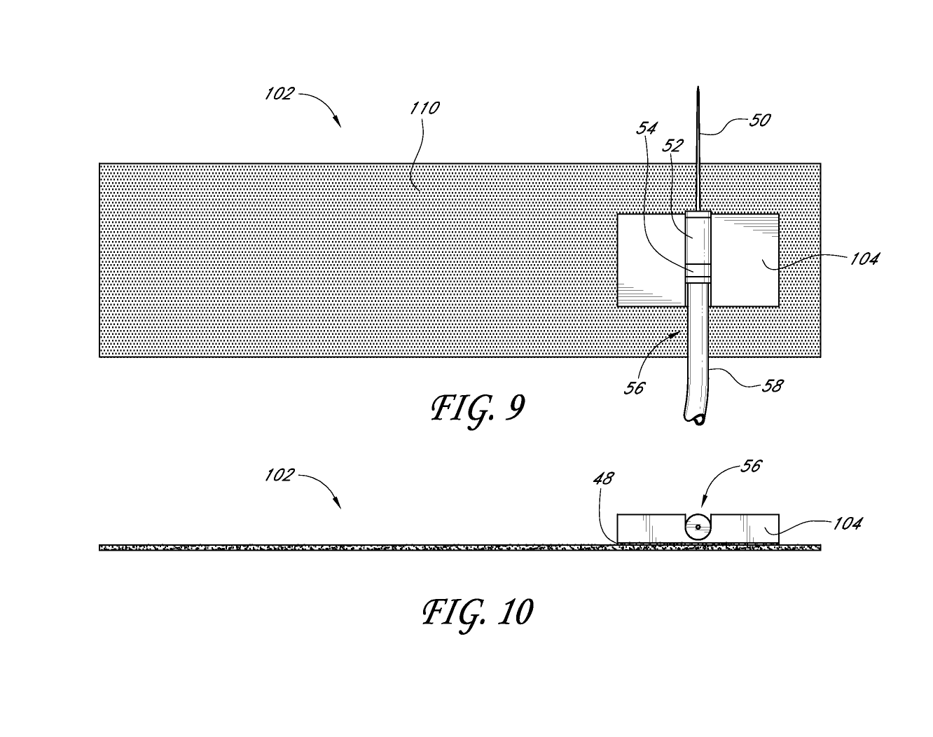

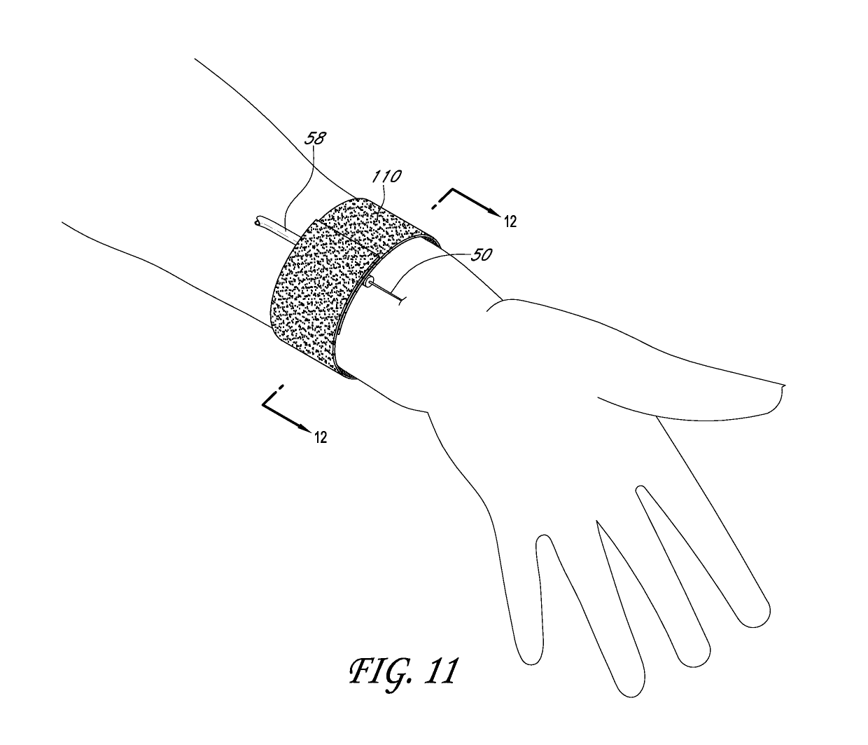

Reference is now made to FIGS. 1-12, wherein like numerals are used to designate like parts throughout. FIG. 1 illustrates a securement system 100, including a strap 102 with a resilient retainer 104 secured thereon for securing a medical article, such as a catheter, and the like to a body or body part of a patient. As shown in FIG. 2, the strap 102 of the securement system 100 can include means for fastening the strap to the body, limb, or body part, including hook 108 and loop 110 fasteners, for example, Velcro® material. As shown in FIG. 7, the resilient retainer 104 secures the medical article 56 while at the same time adhering to the body or body part of the patient. Any suitable techniques for packaging the securement system 100, along with gels, sprays, and the like, can be employed, as will be appreciated by those skilled in the relevant art(s).

The resilient retainer 104 can be configured to be “tacky,” meaning that the resilient retainer has an adhesive property that is somewhat sticky to the touch, enabling the resilient retainer 104 to be readily attached to the medical article 56 and to a limb or other area of a patient's body, yet be easily removed. The resilient retainer 104 may optionally be secured to the strap 102 using an adhesive 48. In one embodiment, the resilient retainer 104 is secured to the strap 102, for example, using any suitable adhesive or is secured without use of an adhesive by configuring the securement surface of the resilient retainer 104 to have a suitable tackiness for securement to the strap 102.

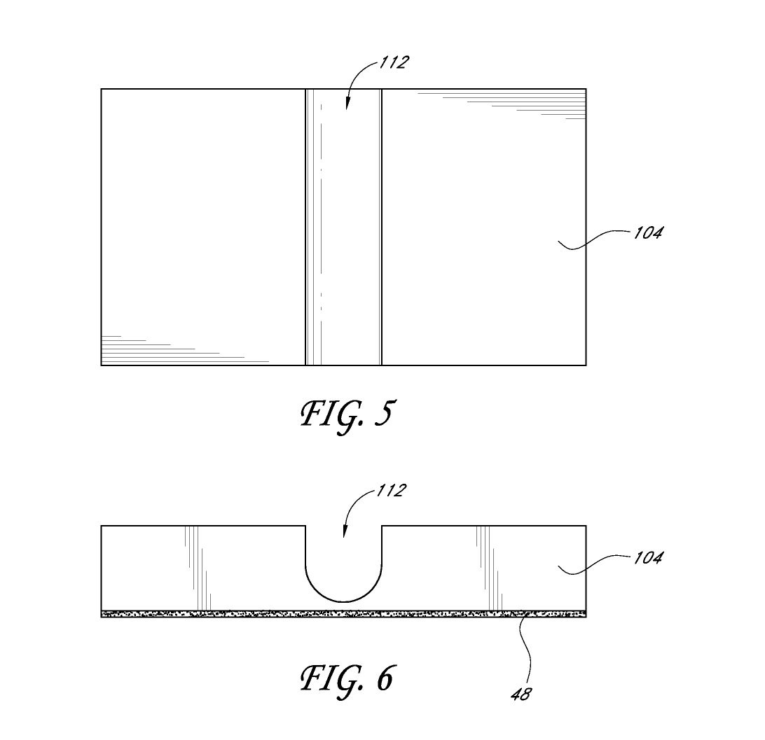

In an exemplary form, the general structure of the resilient retainer 104 can be configured in various shapes, such as rectangular, oval shape, circular, trapezoidal, square, and the like, although other shapes can be employed depending upon the particular application, as will be appreciated by those skilled in the relevant art(s). As shown in FIGS. 5 and 6, the resilient retainer 104 may include a channel 112.

The channel 112 is capable of receiving at least a portion or length of the medical article and is generally configured to house, or grip, and to secure this portion of the medical article. In the form illustrated herein in FIGS. 1 through 12, the channel 112 has a generally semi-circular cross-sectional shape. However, those skilled in the art will understand that the channel 112 can have a different cross-sectional shape. An inner surface contour of the channel 112 is selected depending on the geometry of the portion of the medical article to be retained. For example, in a retainer 104 that is configured to retain a portion of a medical article that has a constant outer diameter, the channel 112 has a constant radius along its length. In contrast, in a retainer 104 configured to retain a portion of a medical article that has a tapering outer surface, the channel 112 may have a tapering inner surface and a radius that varies along the channel length.

Additional embodiments of the channel 112 of the retainer 104 can comprise a plurality of different radii and/or tapering regions. For example, as disclosed in U.S. Pat. No. 7,014,627, the contents of which are hereby incorporated by reference in their entirety, the channel 112 can have two sections: a first proximal section having a generally uniform cross-sectional size along its length while a second distal section has a tapering shape along its length. An abutment wall may be provided to form a transition between these two sections of the channel. These sections of the channel 112 can also both be tapered or straight (i.e., have a generally uniform radius along the length of the section) or the distal section can be straight and the proximal section can be tapered. In this way, the size and shape of the channel 112 can be chosen to match or to approximate the size and shape of the medical article or portion thereof, e.g., the catheter hub, to be retained.

By matching the inner surface contour of the channel 112 to the outer surface of the secured portion of a medical article or device, a more effective securement may be achieved. In addition or in the alternative, effective securement can also be achieved by the engagement of one or more abutment surface of the retainer 104 with one or more contact surfaces on the medical article. Each abutment surface can cooperate with a contact surface on the medical article to inhibit movement of the medical article relative to the retainer. In one embodiment, the medical article may comprise a winged catheter with one or more contact surfaces on the wings.

Although the channel 112 can be formed in various shapes depending upon the desired application (e.g., depending upon the shape of the retained portion of the medical article for which the retainer is designed to be used), the channel 112 desirably has a sufficient length in the longitudinal direction to stabilize the connector fitting, catheter hub, or other medical article, rather than act as a fulcrum for the fitting, as mentioned above. That is, the retainer 104 receives a sufficient length of the medical article 56 to inhibit movement of the medical article in the lateral, longitudinal and transverse direction (i.e., to inhibit yaw, pitch and axial movement of the article).

As shown in FIG. 7, the resilient retainer 104 may include one or more abutment surfaces 60. To arrest longitudinal motion in the illustrated embodiment, a contact surface in the form of an outwardly extending member or tab (not shown) may be employed on the medical article. The outwardly extending member may abut the abutment surface 60 of the retainer 104 to inhibit longitudinal movement in the distal direction. The medical article may include another outwardly extending member that abuts another surface of the retainer 104 to inhibit movement in another direction.

Referring now to FIGS. 5 and 6, in one form, the resilient retainer 104 may be a gel pad produced from a high coefficient of friction, tacky, gel-like material. In one form, retainer 104 may be a cured, tacky, reinforced polysiloxane elastomer. Retainer 104 may be formed by curing a mixture of a lower alkenyl-functional polysiloxane, such as a vinyl containing polysiloxane, and a hydrogen containing polysiloxane copolymer containing active hydrogen groups. In this regard, the term “hydrogen” refers to active hydrogen that is directly bonded to a silicon atom (Si—H), for example, silicon hydrides and hydrogen containing organopolysiloxanes. Such amounts of the hydrogen containing polysiloxane copolymer will be dependent upon factors such as the molar ratio of alkenyl radicals to active hydrogen in the uncured composition and the nature of these components, including such variables as polymer chain length, molecular weight and polymer structure.

The organopolysiloxane elastomers disclosed herein, prior to curing, have a ratio of hydrogen to alkenyl radicals of less than 1.5, or 0.5 to 1.2, which imparts tack or tackiness to the end product produced therefrom. The tackiness is believed to be caused by the partially crosslinked organopolysiloxane elastomers.

It should be recognized that the tacky retainer 104 may possess the requisite tack property throughout the entire retainer. However, surface tack can be modified to be greater than or less than the interior tack. Quantitative measurements of tackiness can be made using a suitable tack tester, such as a Polyken® probe tack tester, a rolling ball tack tester, a peel tester or combinations thereof. Tack can be tested with the Polyken® probe tester in accordance with any suitable procedure, such as American Society For Testing and Materials (ASTM) Designation: D2979-71 (Reapproved 1982), Standard Test Method for Pressure-Sensitive Tack of Adhesives Using an Inverted Probe Machine, pp. 187-189, from the Annual Book of ASTM Standards, Vol. 15.09. The Polyken® probe tack tester is the trademark of the Kendall Company, under license by Testing Machines Inc., Mineola, Long Island, N.Y. Tack can also be tested with a rolling ball tack tester in accordance with Pressure Sensitive Tape Council, Test Methods for Pressure Sensitive Tapes, 9th Edition, PSTC-6, revised August 1989, pp. 29-30 or ASTM D3121. Tack can also be tested with a peel tester in accordance with Pressure Sensitive Tape Council, Test Methods for Pressure Sensitive Tapes, 9th Edition, PSTC-1, revised August 1989, pp. 21-22. The tacky, cushioning layer can be artificially aged prior to tack testing using conventional accelerating aging procedures, such as by exposing the layer to ultraviolet light, elevated temperatures and/or elevated humidity.

The tacky retainer disclosed herein has little or no ability to induce maceration of the skin, due in part, to its permeability for transporting water vapor from the skin through the retainer. Thus, the tacky layer disclosed herein can provide a third, tri-function of inducing little or no maceration when applied to the skin for an extended period. One test method for evaluating water vapor transmission is ASTM Designation: E96-80, Standard Test Methods for Water Vapor Transmission of Materials, edited May 1987, pp. 629-633.

Determinations of the hardness of the retainer 104 can be made with any suitable durometer for testing hardness. One test method entails resting the edge of a Shore 00 durometer on a material, applying a presser foot to the material without shock and taking the average of three readings. Further details for testing hardness can be found in ASTM Test Method D2240. One of ordinary skill in the art will appreciate that elastomers measured by the Shore 00 durometer scale are softer than those measured by the Shore A durometer scale.

Representative vinyl-containing high viscosity organopolysiloxanes of formula (1) suitable for preparing a base material include, but are not limited to the following.

| | |
|---|---|
| | (1) |
| 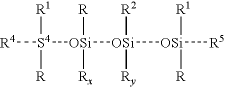 | |

| Polymer | R | R² | R³ | R⁴ | R⁵ | x | y |
|---|---|---|---|---|---|---|---|
| 1 | —CH₃ | —CH₃ | —C₆H₅ | —CH₃ | —C₂H₃ | 925 | 50 |
| 2 | —CH₃ | —CH₃ | —C₆H₅ | —C₂H₃ | —C₂H₃ | 809 | 45 |
| 3 | —CH₃ | —CH₃ | —C₆H₅ | —C₂H₃ | —C₂H₃ | 611 | 41 |
| 4 | —CH₃ | —CH₃ | —C₆H₅ | —C₂H₃ | —C₂H₅ | 471 | 30 |
| 5 | —CH₃ | —CH₃ | —CH₃ | —C₂H₃ | —CH₃ | 600 | 20 |
| 6 | —CH₃ | —CH₃ | —CH₃ | —C₂H₃ | —C₂H₅ | 600 | 20 |

Representative low viscosity organopolysiloxanes of formula (2) suitable for use in preparing a base material include, but are not limited to the following.

| | | | | | | | | (2) |
|---|---|---|---|---|---|---|---|---|
| 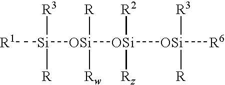 | | | | | | | | |
| Polymer | | R | R² | R³ | R⁴ | R⁶ | x | z |
| 1 | —CH₃ | —C₂H₃ | —C₆H₅ | —CH₃ | —CH₃ | 138 | 13 | |
| 2 | —CH₃ | —C₂H₃ | —C₆H₅ | —CH₃ | —CH₃ | 192 | 39 | |
| 3 | —CH₃ | —C₂H₃ | —CH₃ | —CH₃ | —CH₃ | 125 | 25 | |
| 4 | —CH₃ | —CH₃ | —CH₃ | —CH₃ | —CH₃ | 90 | 20 | |
| 5 | —CH₃ | —CH₃ | —CH₃ | —CH₃ | —CH₃ | 125 | 25 | |

The base material prepared from the vinyl-containing high viscosity organopolysiloxanes of formula (1) and the low viscosity organopolysiloxanes of formula (2) can be admixed with a copolymer containing dimethyl and methyl hydrogen siloxanes. The amount of hydrogen-containing organopolysiloxane used should be sufficient to achieve a ratio of alkenyl radicals to hydrogen in the uncured composition of less than 1.2.

The elastomers are reinforced with a suitable reinforcing agent or filler such as titanium dioxide, calcium carbonate, lithopone, zinc oxide, zirconium silicate, silica aerogel, iron oxide, diatomaceous earth, silazane-treated silica, precipitated silica, fumed silica, mined silica, glass fibers, magnesium oxide, chromic oxide, zirconium oxide, aluminum oxide, alpha quartz, calcined clay and the like, as well as various reinforcing silica fillers taught in U.S. Pat. No. 3,635,743, the contents of which are hereby incorporated by reference, or mixtures of any of the above, or a filler selected from silazane treated silica, precipitated silica and fumed silica or mixtures thereof. In one form, the reinforcing filler is a highly reinforcing silica filler with a surface area ranging from about 80 to about 400 square meters/gram (m2/g), or from about 200 to about 400 m2/g. Typically the reinforcing agent is mixed with the vinyl-containing high viscosity organopolysiloxane (1) and low viscosity organopolysiloxane (2) prior to addition of the hydrogen containing polysiloxane copolymer. The reinforcing filler can be employed in the uncured composition in an amount ranging from 10 to about 70 parts per 100 parts of the uncured composition, or from 15 to about 40 parts, or from about 20 to about 30 parts. In the cured tacky, reinforced cushioning layer, such amounts correspond to about ten to about 70% by weight, or from about 15 to about 40%, or from about 20 to about 30%.

The durometer or hardness of the polysiloxane elastomers disclosed herein can be lowered (i.e. made softer) by incorporating low viscosity polysiloxanes into the uncured composition. Representative low viscosity polysiloxanes include polydimethylsiloxane fluids or vinyl-containing polydimethylsiloxane fluids. The molecular weight average of the plasticizer can range from about 750 to about 30,000. The low viscosity polysiloxanes can be employed in an amount ranging from about zero to about 50% by weight of the uncured composition, or from about 10 to about 30%.

The polysiloxane elastomers disclosed herein possess suitable hardness, tensile strength, elongation and tear strength, as based upon standard elastic materials testing. Unreinforced polysiloxane compositions must be enclosed in an envelope or other supporting means, i.e. foam impregnation, in order to maintain the shape or durability of an article produced therefrom. In contrast, the high coefficient of friction, tacky, polysiloxane retainer 104 disclosed herein is viscoelastic and has a measurable hardness, tensile strength, elongation and/or tear strength.

Further, the tacky, reinforced polysiloxanes disclosed herein can retain their elastic properties after prolonged action of compressive stresses, a property known as compression set. Compression set is an indicator of durability. According to ASTM Designation: D395-85, Standard Test Methods for Rubber Property Compression Set, pp. 34-35, the actual stressing service may involve the maintenance of a definite deflection, the constant application of a known force, or the rapidly repeated deformation and recovery resulting from intermittent compressive forces. Though the latter dynamic stressing, like the others, produces compression set, its effects as a whole are simulated more closely by compression flexing or hysteresis tests. Therefore, compression set tests are considered to be mainly applicable to service conditions involving static stresses.

Tests are frequently conducted at elevated temperatures. In a first method utilizing static stresses, a test specimen is compressed to a deflection and maintained under this condition for a specified time and at a specified temperature. In a second method utilizing static stresses, a specified force is maintained under this condition for a specified time and at a specified temperature. After application of the specified deflection or specified force the residual deformation of a test specimen is measured 30 minutes after removal from a suitable compression device in which the specimen has been subjected for a definite time to compressive deformation under specified conditions. After measurement of the residual deformation, the compression set as specified in the appropriate method is calculated according to ASTM D395-85 equations.

When produced in accordance herewith, retainers 104 may be prepared to exhibit the following physical properties: a durometer hardness of from about 5 to 55 units (Shore 00), a tensile strength of from about 20 to about 800 psi, a minimum elongation of from about 250 to about 1100 percent, a tear strength of from about 5 to about 200 lb/in, a polyken probe tack of about 10 to about 450 grams, a rolling ball tack of about 0 to about 3 inches and a peel test value of from about 0.02 to about 80 lb/in. Of course, the retainer 104 is not limited to the above described properties.

The retainer 104 can be prepared using techniques such as molding, liquid injection molding, transfer molding, casting and the like. Retainer 104 can be preformed into a desired shape for use with securement system 100, supplied in a sheet form, which may be cut to the desired shape prior to use or the gel material provided in a kit form, wherein the catalyst is provided in a first container and the other components premixed and provided in a second container. In the latter case, a mold is provided and the components mixed, poured into the mold and cured. Curing can be with or without heat.

Such curing can be achieved by increasing the molecular weight of the uncured polysiloxane elastomers to the extent desired through crosslinking, using heating or standing at ambient, as described U.S. Pat. No. 3,445,420. Generally, the degree to which the uncured polysiloxane composition can be partially crosslinked can range from about 30 to about 90%, based upon the alkenyl-containing polysiloxane, or from about 30 to about 60%.

In another form, foam may be employed as resilient retainer 104. In one form, the foam is memory foam made from polyurethane with additional chemical additives that add to its viscosity level, thereby increasing the density of the foam, as those skilled in the art will plainly recognize. This type of memory foam is often referred to as viscoelastic polyurethane foam. Depending on the chemicals used and the overall density of the foam, it can be firmer in cooler temperatures and softer in warmer environments. As may be appreciated, higher density memory foam will react with body heat to allow it to mold itself to the shape of a warm body within a few minutes. However, a lower density memory foam is pressure-sensitive and will mold more quickly to the shape of the body. The example often used for a demonstration of its properties is a hand pressed into the foam and then removed, leaving a clear impression of the hand in the foam.

In recent years, viscoelastic memory foam has become cheaper to produce and is now widely available, lending to its application herein. Its heat-retaining properties are also helpful to some pain sufferers, who find the added warmth also helps alleviate pain.

In one form, the memory foam is selected from non-hydrophilic polyurethane memory foam and hydrophilic polyurethane memory foam. When a non-hydrophilic polyurethane memory foam is employed, it may be prepared by mixing about 0.5 to about 90 wt % of polyether polyol with about 2 to about 60 wt % isocyanate at room temperature and foaming the mixture with a foaming agent selected form methylene chloride and carbon dioxide in a mold.

When a hydrophilic polyurethane memory foam is employed, it may be prepared by foaming a starting material, the starting material comprising about 1 to about 70 wt % of a hydrophilic polyurethane prepolymer; about 1 to about 50 wt % of an acrylic emulsion polymer; water and a surfactant. The polyurethane prepolymer may include about 1 to about 80 wt % of a first polyether polyol and about 1 to about 70 wt % of isocyanate. In one form, the hydrophilic polyurethane memory foam may be foamed using carbon dioxide, which is generated upon reaction of the isocyanate and the water.

When produced in accordance herewith, the foam resilient retainer 104 can be prepared to exhibit the following physical properties: a durometer hardness of from about 5 to 55 units (Shore 00), a tensile strength of from about 20 to about 800 psi, a minimum elongation of from about 250 to about 1100 percent, a tear strength of from about 5 to about 200 lb/in, a polyken probe tack of about 10 to about 450 grams, a rolling ball tack of about 0 to about 3 inches and a peel test value of from about 0.02 to about 80 lb/in. Of course, the retainer 104 is not limited to the above described properties.

Although the exemplary forms are described in terms of use with hook 108 and loop 110 fasteners, such as Velcro®, and the like, the exemplary forms can be employed with any other suitable fasteners, such as snaps, ties, and the like, as will be appreciated by those skilled in the relevant art(s).

### Medical Articles

Although the exemplary forms are described in terms of use with medical devices, such as catheters, and the like, the exemplary forms can be employed with any other suitable medical devices, as will be appreciated by those skilled in the relevant art(s).

An exemplary medical article for use with the embodiment of the securement device described above will now be described with reference to FIGS. 7-12. The medical article 56 can be a single medical article or a combination of one or more medical articles. Such medical articles can be or include, for example, but without limitation, connector fittings, catheters, catheter hubs, catheter adaptors, fluid supply lines, or other similar articles. In one embodiment, the medical article 56 can comprise a winged catheter. FIG. 7 shows in a perspective view a catheter hub 52 and a connector fitting 54. The connector fitting 54 may be disposed upon the end of a medical line 58 which can be connected to a drip bag, blood monitor, or other fluid related medical apparatus. While the retainer 104 is configured to receive a portion of the catheter hub 52, the retainer can be configured for use with the connector fitting 54, as will be described.

The connector fitting 54 comprises an elongated body which is attached to the end of the medical line 58. A tapered portion (not shown) of the connector fitting 54 also may include a centrally disposed lumen that communicates with the lumen of the medical line.

As indicated, FIG. 7 shows connector fitting 54 secured in the proximal position and secured to the catheter hub 52. When the connector fitting 54 is inserted into the catheter hub 52, the lumen of the connector fitting is disposed in fluid communication with the lumen of the catheter hub 52. This provides fluid communication between the medical line 58 and the patient. The connector fitting 54 may optionally include contact surfaces in the form of an outwardly extending element or member. In one embodiment, the connector fitting 54 may comprise an outwardly extending tab. The retainer 104 can be used with both luer slip and luer lock connector fittings, as those skilled in the art will plainly recognize.

### Operation

An exemplary process for coupling a medical article with securement system 100 described above will now be described with reference to FIGS. 1-12.

In one form, provided is a method of securing a medical article to a patient. The method includes the step of providing an apparatus that includes a securement strap and a resilient retainer. The securement strap has an inner surface and an outer surface. The retainer is disposed on the inner surface and has a channel and channel opening arranged along a longitudinal axis. The method also includes the steps of placing at least a portion of the medical article within the longitudinal channel and securing the apparatus and medical article to the patient with the securement strap.

One method of using the securement system 100 will be described in the context of starting an intravenous line. However, the aspects and features of the operational method and the use of the present securement system 100 are not limited to this particular application.

A healthcare provider begins the procedure by inserting an IV catheter into a patient's vein in a known manner and then attaching an intravenous line to the IV catheter though the luer connection. In particular, the healthcare provider inserts the tapered or luer end of the connector fitting 54 into the catheter hub 52. This action draws together the two medical article components and releasably interlocks them. The immediate connection of the IV line to the catheter inhibits a back flow of blood through the catheter. The healthcare provider now secures the IV catheter in place on the patient using the securement system 100. In some variations of this method, however, the securement system 100 can be first be attached to one or both of the medical article (as well as the possibly to the patient) before the healthcare provider makes the connection between the two medical articles.

In order to illustrate more clearly the interaction between the retainer 104, the catheter hub 52, and the securement strap 102 of securement system 100, reference is made to FIG. 7 wherein an exploded perspective view of the securement system 100 is illustrated.

In use, the healthcare provider can secure a medical line 58 and the medical article 56 to a patient using the above-described securement system 100 or a modification thereof. The healthcare provider aligns the medical article 56 at least partially within the retainer 104.

As shown in FIG. 7, a portion of the medical article 56 is inserted into the retainer 104. The medical article 56 is received within the retainer channel and adheres to the tacky surface of the retainer 104. Depending on the diameter of the medical article 56, the channel 112 can provide a snap-fit connection between the medical article 56 and the retainer 104. The medical article 56 may optionally include contact surfaces (not shown) that meet abutment surface 60 on the retainer 104. The combination of the channel shape 112 and the tackiness of the retainer 104 arrests longitudinal, lateral, and transverse movement of the retained section of the medical article 56.

Once the medical article 56 is received within the retainer 104, the tacky retainer, described above, is preferably positioned at the desired location upon the patient (e.g., on the patient's skin) with the medical article 56 disposed at least partially between the retainer 104 and the patient. In some embodiments, the medical article 56 may first be inserted in or placed on the patient and the medical article 56 may then be secured by the retainer 104. The medical article 56 may comprise a needle 50 or other tapered lumen for insertion into the patient via the insertion site. Preferably, the insertion site is uncovered by the retainer 104.

Once the medical article 56 is received within the retainer 104 and the retainer 104 is positioned at the desired location upon the patient, the strap 102 is optionally wrapped around the limb of the patient to secure the retainer 104 to the patient. The strap 102 may be fastened to itself using hook 108 and loop 110 fasteners, for example, Velcro® material. In other embodiments, the strap 102 may be fastened to itself using different mechanisms including snaps, buckles, clips, latches, etc. The strap 102 and tacky retainer 104 combine to secure the medical article 56 to a particular position on a patient. While a single strap 102 is shown, it is also contemplated that a pair of securement straps may be employed to secure the retainer 104 and medical article 56 to the patient,

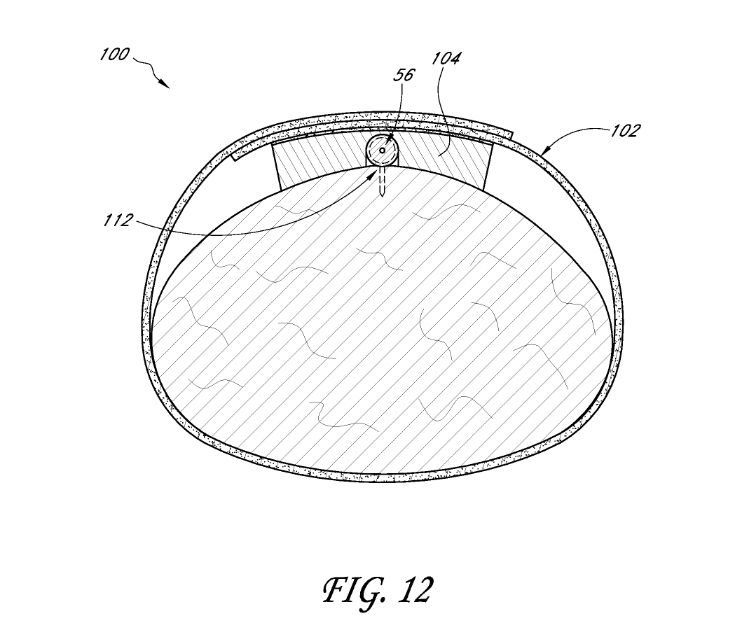

As shown in FIG. 12, when receiving a medical article 56 having a cross-sectional area that is larger than the channel 112, the retainer 104 may compress such that the surface of the channel matches the shape of the received portion of the medical article 56. Additionally, when positioned on the patient, the retainer 104 may self-adhere to the patient and compress to match the contours of the patient's skin due to the tackiness of the retainer material. The strap 102 may also compress the retainer 104 so that its lower surface substantially matches the contours of the patient's skin when the strap is used to secure the retainer against the patient. The compressibility of the retainer 104 allows the retainer to be positioned upon various non-planar surfaces while securing various medical articles 56 relative to the non-planar surfaces.

In other embodiments, the securement strap 102 is not included as part of the securement system 100. For example, the tacky retainer 104 may be configured to adhere to the skin of a patient without a strap. As may be appreciated, the channel 112 of the retainer 104 surrounds an arc length of more than 180 degrees of the medical article 56. This inhibits transverse or lateral motion of the medical article relative to the retainer 104 and the insertion site.

All patents, test procedures, and other documents cited herein, are fully incorporated by reference to the extent such disclosure is not inconsistent with this disclosure and for all jurisdictions in which such incorporation is permitted.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the disclosure and the claims that follow.

What is claimed is:

1. A securement system for use with an elongated medical article, comprising:
an elongated flexible securement strap having an inner surface and an outer surface, the strap being configured to wrap around a portion of a patient, at least a portion of the inner surface contacting a patient's skin; and
a retainer disposed on at least a portion of the inner surface of the strap and having a channel and channel opening arranged along a longitudinal axis, at least a portion of the retainer being compressible so as to substantially match contours of the patient's skin and of the secured medical article at least when the strap is secured around the portion of the patient.

2. The securement system of claim 1, wherein the retainer receives a sufficient length of the medical article to inhibit movement of the medical article in the lateral, longitudinal and transverse directions at least when the strap is secured around the portion of the patient.

3. The securement system of claim 1, wherein the medical article is an IV catheter.

4. The securement system of claim 1 further comprising an adhesive disclosed between the retainer and the inner surface.

5. The securement system of claim 1, wherein the retainer comprises a material that has tacky properties.

6. The securement system of claim 5 further comprising an elastomeric gel.

7. The securement system of claim 6, wherein the elastomeric gel is formed by curing an organopolysiloxane composition.

8. The securement system of claim 7, wherein the organopolysiloxane composition comprises:
a vinyl-containing high viscosity organopolysiloxane or a blend of high viscosity vinyl-containing organopolysiloxanes;
a low viscosity organopolysiloxane or a blend of low viscosity organopolysiloxanes;
a reinforcing filler;
a platinum catalyst; and
a hydrogen containing polysiloxane copolymer.

9. The securement system of claim 8, wherein the molar ratio of hydrogen to vinyl radicals in the total composition is less than 1.2, such that after curing, the degree to which the tacky, reinforced polysiloxane elastomer is partially crosslinked is about 30 to about 90%.

10. The securement system of claim 7, wherein the organopolysiloxane composition has a hardness of about 5 to about 55 durometer units (Shore 00), a tackiness of about 0 to about 450 grams as determined by a polyken probe tack tester or about 0 to about 7.6 cm (about 0 to about 3 inches) as determined by a rolling ball tack tester and a tensile strength of about 0.14 to about 5.52 mega Pascals (about 20 to about 800 pounds/square inch), a minimum elongation of about 250 to about 1100 percent and a tear strength of about 0.8 to about 35.2 kN/m (about 5 to about 200 pound/square inch).

11. The securement system of claim 7, wherein the organopolysiloxane composition comprises, based upon 100 parts total composition:
20 to 90 parts of a vinyl-containing high viscosity organopolysiloxane or a blend of high viscosity vinyl-containing organopolysiloxanes having no more than 25 mole percent of phenyl radicals and having a viscosity of 2,000 to 1,000,000 centipoise at 25° C. of the formula: 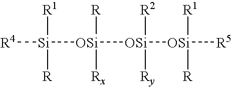
where R¹ is selected from the class consisting of alkenyl, alkyl and aryl radicals and R is a monovalent hydrocarbon radical, R² is selected from the class consisting of alkyl and aryl radicals, R⁴ and R⁵ are independently selected from the class consisting of alkyl and vinyl radicals; x varies from zero to 3000; and y varies from 0 to 300;
from 5 to 40 parts of a polymer selected from the class consisting of a low viscosity organopolysiloxane and a blend of low viscosity organopolysiloxanes having viscosity that varies from 20 to 5,000 centipoise at 25° C. and having no more than 25 mole percent phenyl radicals of the formula: 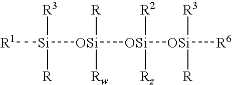
wherein R¹ and R⁶ are independently selected from the class consisting of alkenyl, alkyl and aryl radicals, R² and R are as previously defined, R³ is selected from the class consisting of alkyl, aryl and alkenyl radicals, w varies from 0 to 500, and z varies from 0 to 200;
from 10 to 70 parts of a reinforcing filler;
from 0.1 to 50 parts per million of platinum catalyst (as platinum metal) to the total composition; and
from 0.1 to 50 parts of a hydrogen containing polysiloxane copolymer, wherein the molar ratio of hydrogen to alkenyl radicals in the total uncured composition is less than 1.2, such that after curing, the degree to which the soft, tacky, reinforced polysiloxane elastomer is partially crosslinked is about 30 to about 90%.

12. The securement system of claim 1, wherein the securement strap is releasably attachable to the patient.

13. The securement system of claim 12, wherein the securement strap comprises hook and loop fasteners.

14. The securement system of claim 13, wherein the securement strap comprises Velcro® material.

15. The securement system of claim 12, wherein the securement strap comprises snaps.

16. The securement system of claim 1 further comprising memory foam.

17. The securement system of claim 16, wherein the memory foam is selected from non-hydrophilic polyurethane memory foam and hydrophilic polyurethane memory foam.

18. The securement system of claim 17, wherein the non-hydrophilic polyurethane memory foam is prepared by mixing about 0.5 to about 90 wt % of polyether polyol with about 2 to about 60 wt % isocyanate at room temperature and foaming the mixture with a foaming agent selected form methylene chloride and carbon dioxide in a mold.

19. The securement system of claim 17, wherein the hydrophilic polyurethane memory foam is prepared by foaming a starting material, the starting material comprising about 1 to about 70 wt % of a hydrophilic polyurethane prepolymer; about 1 to about 50 wt % of an acrylic emulsion polymer; water and a surfactant; wherein the polyurethane prepolymer comprises about 1 to about 80 wt % of a first polyether polyol and about 1 to about 70 wt % of isocyanate; whereby the hydrophilic polyurethane memory foam is foamed using carbon dioxide, which is generated upon reaction of the isocyanate and the water.

20. A method of securing a medical article to a patient, comprising:
providing an apparatus, the apparatus including a securement strap having an inner surface and an outer surface, and a retainer disposed on at least a portion of the inner surface of the strap and having a longitudinal channel configured to receive and retain at least a portion of the medical article;
placing at least a portion of the medical article within the longitudinal channel; and
securing the retainer and medical article to the patient with the securement strap so that the retainer is disposed between the patient and the securement strap and the secured portion of the medical article is inhibited from moving in the lateral, longitudinal and transverse directions relative to the patient.

21. The method of claim 20, wherein the retainer is formed from an elastomeric gel.

22. The method of claim 21, wherein the retainer is formed by curing an organopolysiloxane composition.

23. The method of claim 20, wherein the retainer comprises memory foam.

24. The method of claim 20, wherein the memory foam is selected from non-hydrophilic polyurethane memory foam and hydrophilic polyurethane memory foam.

25. The method of claim 20, wherein the medical article is an IV catheter.

26. A retainer for use with an elongated medical article, comprising:
a channel sized and shaped to receive at least a portion of the medical article;
a channel opening arranged along a longitudinal axis and extending through a bottom surface of the retainer; and
an elastomeric gel formed by curing an organopolysiloxane composition, the gel being compressible so as to substantially match contours of a patient's skin and of the medical article at least when the bottom surface is urged against the patient's skin.
